SEQUENCE LISTING (4 sequences)

SEQ ID NO: 1 - DNA, 1301 bp, Calendula officinalis
  CDS (21)...(1142)
gctacacctagctacgtaccatgggcaaaggagcatcaaacaagaaggttttggaacgagttccaatcacaaaaccgccattcgaatacaatgatctgaagaaagcagtaccaccacattgtttttcacgaccacttttccgttcgttttatttcctacttcacgacattattgtaacatgtatccttttctacgtagcatcaaactacattcctatgctccctggtttcctttcctacattgtatggcctgtttactggatctcccaaggagtttttcttggcagattgtggatgattggccatgaatgcggccatcatagttttagtaattaccgttgggtcgacgatagtgttggttttttaatccatacggccaccctcactccctatttttccttcaaatatagtcaccgtaatcaccatgcacacaccaattccatggaatatgacgaagttcatatcccgaaacgcaaatccgaagctctagatctctactttgaatttctcggcaacaacccgatggggttaatgatcaccatgttatgtaaactcacttttggatatgcagcttacattatgttcaattatacaggcaagaagcacaaatctgggggtttagcaagtcacttctacccacaaagccctctctttaacgacagcgaacgtaatcatgttttgttctctgatgtcgggatttgcatcgtcttgtacgcatgttaccgtattgtgatggtcacaggggcaatgtcggcattttatgtgtacggcattccttgggttataatgagtgctattctctttgcagcaacttatttacaacacactcatccttcgatccctcattatgatacaactgagtggaactggcttagaggggcattatcgacaattgatagagatttagggttcttcaacatgaacaaaacacattatcatgttatccaccatttatttcctgtcattccggaataccatgcacaagaggcaactgaggccatcaagcccatcttaggtcaatattacaagtatgatggtactccgtttttaaaggcgttgtggagagaaatgaaggactgtatttatgtagaatccgatcaaggtcagaagaaacaaggtatttactggttcaagaataagatttgaagtttcaaataatctggactacgtttaattttgtgccatcatcagtaccgtgaattagttttgttgtgtttttaattttaatttcgtgtgatggtgtaatgtaatataattcagtataataaaggcgttatcctttcaaaaaaaaaaaaaaaaaaa

SEQ ID NO: 2 - protein, 374 aa, Calendula officinalis
MGKGASNKKVLERVPITKPPFEYNDLKKAVPPHCFSRPLFRSFYFLLHDIIVTCILFYVASNYIPMLPGFLSYIVWPVYWISQGVFLGRLWMIGHECGHHSFSNYRWVDDSVGFLIHTATLTPYFSFKYSHRNHHAHTNSMEYDEVHIPKRKSEALDLYFEFLGNNPMGLMITMLCKLTFGYAAYIMFNYTGKKHKSGGLASHFYPQSPLFNDSERNHVLFSDVGICIVLYACYRIVMVTGAMSAFYVYGIPWVIMSAILFAATYLQHTHPSIPHYDTTEWNWLRGALSTIDRDLGFFNMNKTHYHVIHHLFPVIPEYHAQEATEAIKPILGQYYKYDGTPFLKALWREMKDCIYVESDQGQKKQGIYWFKNKI

SEQ ID NO: 3 - DNA, 1411 bp, Calendula officinalis
  CDS (89)...(1237)
gcgaacgccgaacggccatttcactcttccgatttgaagaaacaaatagcagattcaactctcaaatcgtctttcaggtcattgaacgatgggtgcaggcggtcgaatgcaagatcccaccaacggtggcaacaaaaccgagcccgaaccaatccaacgggtcccacatgaaaaacccccattcacagttggagacatcaagaaagcgatcccacctcattgtttcaaccgatcggtaattcgttcattttcatacgtcttttacgacctcacaatcgcgtcaatcttgtactacattgccaacaattacatctctaccctccctagcccgctcgcctacgtggcatggcccgtttactgggccgtccaagggtgcgtcttaaccggggtgtgggtcatagcccacgaatgtggccatcatgcttttagcgaccaccaatggctcgatgacaccgtgggtctcgtcttgcactcgttcctactcgtgccctacttttcgtggaaatatagccaccgtaggcaccactcgaacacgggctcgatcgagcacgatgaggttttcgtcccgaagttgaaatcgggcgtccggtcaaccgcccggtacctaaacaacccaccgggccgaatcttgaccctactcgtaaccctaaccctcggttggcctctatacctcacgttcaacgtttcgggccgttactacgaccggttcgcgtgccatttcgacccgaatagcccgatctactcgaagcgcgaacgggctcaaatcttcatatccgacgccgggatcttagccgtagtcttcgtactcttccgactcgcaatgaccaaagggctcacgtgggtcctaaccatgtacggtggcccgttactcgtggtcaacggtttcctagtcttgatcacattcctacaacacactcacccttcgctcccgcactatgactcaaccgaatgggattggttacgtggggccctcaccacaatcgaccgtgattacgggatcctaaacaaagtgttccataacataaccgacactcacgtggcccaccatttgttctctacaatgcctcattaccatgcaatggaagccacgaaggtgatcaaaccgattttgggcgattattatcagtttgacgggacctcgatttttaaggcgatgtatcgggaaacaaaggagtgcatttatgttgataaggatgaggaggtgaaagatggtgtttattggtatcgtaataagatttaagagaatggcaaatctgtattcctttttaagtggtgttatttagtgtatgtttggtctttttaagacagttatgaatggtagtcggatgaataactttgtatccgaactgtgatgtttggtttagcttcttaaaatgggttgactttagcttccaaaaaaaaaaaaaaaaaa

SEQ ID NO: 4 - protein, 383 aa, Calendula officinalis
MGAGGRMQDPTNGGNKTEPEPIQRVPHEKPPFTVGDIKKAIPPHCFNRSVIRSFSYVFYDLTIASILYYIANNYISTLPSPLAYVAWPVYWAVQGCVLTGVWVIAHECGHHAFSDHQWLDDTVGLVLHSFLLVPYFSWKYSHRRHHSNTGSIEHDEVFVPKLKSGVRSTARYLNNPPGRILTLLVTLTLGWPLYLTFNVSGRYYDRFACHFDPNSPIYSKRERAQIFISDAGILAVVFVLFRLAMTKGLTWVLTMYGGPLLVVNGFLVLITFLQHTHPSLPHYDSTEWDWLRGALTTIDRDYGILNKVFHNITDTHVAHHLFSTMPHYHAMEATKVIKPILGDYYQFDGTSIFKAMYRETKECIYVDKDEEVKDGVYWYRNKI

(19) United States
(12) Patent Application Publication
(10) Pub. No.: US 2002/0045232 A1
(43) Pub. Date: Apr. 18, 2002

(54) Production of conjugated linoleic and linolenic acids in plants

(76) Inventor: Xiao Qiu, Saskatoon (CA)

Correspondence Address: LAHIVE & COCKFIELD, 28 STATE STREET, BOSTON, MA, 02109, US

(21) Appl. No.: 09/852,399

(22) Filed: May 9, 2001

Related U.S. Application Data
(60) Provisional application No. 60/203,027, filed on May 9, 2000.

(51) Int. Cl. C12N 9/04; C07H 21/04; A01H 5/00; C12N 5/04

(52) U.S. Cl. 435/190; 435/410; 536/23.2; 435/320.1; 800/278

(57) ABSTRACT

The present invention relates to methods for large-scale production of conjugated fatty acids, especially conjugated linoleic acids in plants. More specifically, the present invention relates to genes identified from Calendula officinalis coding for a conjugase and its related enzyme, a Δ12 desaturase, and utilization of them for large scale production of conjugated linoleic and linolenic acids in plants. The genes encoding a conjugase can introduce two conjugated double bonds at 8 and 10 positions, and a Δ12 desaturase can introduce a double bond at 12 position of acyl chains. The constructs containing these genes can be transferred to plants with different substrate profiles, which allows for the production of conjugated linoleic acids (18:2, Δ8, Δ10) and linolenic acids (18:3, Δ8, Δ10, Δ12) in plant seeds on a commercial scale.

### RELATED APPLICATIONS

This application claims priority to U.S. Provisional Application No. 60/203,027, filed May 9, 2000, the entire contents of which are hereby incorporated by reference. The entire contents of Appendix A including the entire contents of all references cited therein also are expressly incorporated by reference and are intended to be part of the present application.

### BACKGROUND OF THE INVENTION

Conjugated fatty acids widely occur in bacteria, algae and plants. A substantial proportion of polyunsaturated fatty acids contain conjugated double bonds, such as a conjugated eicosapentaenioc acid (5Z,8Z,10E,12E,14Z-20:5) and (5Z,7Z,9E,14Z,17Z-20:5) (Burgess et al. (1991) Lipids 26: 162-165; Wise et al. (1994) Biochemistry 33:15223-15232). In plants, conjugated linolenic acid is the most abundant conjugated fatty acid that accumulates in seeds. The examples are α-eleostearic acid (9Z, 11E, 13E-18:3) in Aleurites fordii and Momordica charantia (Liu et al. (1997) Plant Physiol 113:1343-1349), catalpic acid (9E, 11E, 13Z-18:3) in Catalpa ovata, punicic acid (9Z, 11E, 13Z-18:3) in Punica and Cayaponia, and jarcaric acid (8Z, 10E, 12Z-18:3) in Jacaranda (Chisholm et al. (1964) Can. J. Biochem 45:1081-1086; Chisholm et al. (1967) Can. J. Biochem. 45:251-255). Calendula officinalis is an annual flowering plant that can accumulate more than 40% of calendic acid (8E, 10E, 12Z-18:3) in the seeds (Chisholm et al. (1964) Can. J. Biochem 45:1081-1086).

Unlike common unsaturated fatty acids where unsaturation is entirely methylene-interrupted, conjugated fatty acids contain conjugated double bonds in their acyl chains that are not interrupted by a methylene group. As compared to conjugated polyunsaturated acids, conjugated linoleic acids (CLAs) appear less common in nature. Only limited reports have documented the occurrence of these fatty acids. The examples are the foods derived from ruminant animals and a number of anaerobic bacteria such as rumen bacterium Butyrivibrio fibrisolvens (Kepler et al. (1970) J. Biol. Chem. 241(6):1350-1354) and diary starter Propionibacteria. It is believed that CLAs are originally generated by rumen bacteria and then absorbed by host, and eventually distributed in animal products (Pariza et al. (1997) Toxicol. Sciences 52: 107-110).

Conjugated fatty acids, especially CLAs, are a newly recognized type of nutraceutical compound. CLAs have recently drawn tremendous attention of pharmaceutical and nutraceutical industries because of their various physiological effects in animal and humans. (Haumann B. F. (1996) Inform. 7:152159; Pariza et al. (1997) Toxicol. Sciences 52: 107-110). For example, dietary CLA was shown to reduce the development of atherosclerosis in rabbits (Lee et al. (1994) Atherosclerosis 108:19-25) and to inhibit development of various cancers in model animals. It was also reported that CLA is one of the most effective dietary anticarcinogens in animals (Pariza et al (1997) Toxicol. Sciences 52: 107-110). Studies have shown that feeding CLAs at low concentration (0.5% of diet) to rodents and chicken can enhance the immune function (Miller et al (1994) Biopsy. Res. Commun. 198:1107-1112). In addition, CLA was recently found to be able to decrease fat composition, increase lean body mass and improve feed efficiency in chicken and pigs (Pariza et al. (1997) Toxicol. Sciences 52: 107-110; Park et al. (1997) Lipids 32:853-858). It is expected from the finding that CLAs will have potential uses in changing body composition and reducing body mass in human and improving feeding efficiency in animals.

With the growing realization of benefit of CLAs in animal and human, the demand for the product is growing. Unfortunately, there is no rich natural source for this fatty acid. Although some animal foods such as dairy products and meat derived from ruminant contain CLAs, the content is low. The fatty acids account for only about 0.2 to 2.8% of the total fatty acids in the product. Linoleic acid can be converted to CLA by chemical methods (Chen et al. (1999) Lipids 34(8): 879-884). However, CLA derived from chemical process is the mixture of several isomers. The two major isomers (9Z,11E-18:2 and 10E,12Z-18:2) account for about 90% of the product with the proximately equal amount, the rest includes other CLA isomers such as 18:2 (8, 10) and 18:2 (11, 13). Although chemical process is an effective way to produce CLA, there is some potential disadvantage associated with it. First, products derived from the chemical process tend to have highly heterogeneous composition. If a single isomer is needed, extensive purification procedure has to be applied to separate the one from the rest. The purification process can be very expensive because of their similar structure in chemistry. Second, the growing consumer demand for natural products may make synthetic CLA even less desirable in the future.

### SUMMARY OF THE INVENTION

Plant biotechnology has long been considered an efficient way to produce biological compounds. It is cost-effective and renewable with little side effects. Thus, tremendous industrial effort directed to the production of various compounds including speciality fatty acids, biodegradable plastics and pharmaceutical polypeptides through plant biotechnology has ensued. Accordingly, plant biotechnology is an attractive route for producing conjugated fatty acids, especially CLAs, in a safe, cost-efficient manner so as to garner the maximum therapeutic value from these fatty acids.

The present invention is based, at least in part, on the discovery of key enzyme coding genes from Calendula. In particular, the present inventors have identified the CoFad2 (Δ12 desaturase) and CoFac2 (conjugase) genes of Calendula officinalis. The conjugase from Calendula officinalis is an enzyme that can introduce two conjugated double bonds into acyl chain, e.g., it can convert Δ9 double bond of oleic acid and linoleic acids to Δ8 and Δ10 double bonds. The Δ12 desaturase from Calendula officinalis is a desaturase that can introduce a double bond into fatty acids, e.g., at position 12 of oleic acids. The constructs containing the conjugase gene can be used to transform plants that provide high oleic acid, which allows producing CLA (8,10-18:2). The constructs containing the conjugase and Δ12 desaturase genes can be used to transform plants that provide high linoleic acid, which allows producing calendic acid (8,10,12-18:3).

Accordingly, the present invention features methods of producing CLAs and conjugated linolenic acids as well as other conjugated fatty acids. Such methods include, transforming a cell (e.g., a plant cell) with a nucleic acid molecule which encodes a protein having an activity of catalyzing the formation of two conjugated double bonds at position 8 and 10 numbered from the carboxyl end of a fatty acyl chain; transforming a cell with a nucleic acid molecule encoding a protein having an activity of catalyzing the formation of a double bond at position 12 numbered from the carboxyl end of a fatty acyl chain; a method of producing a cell capable of generating conjugated linoleic acid (18:2, Δ8, Δ10) or linolenic acid (18:3, Δ8, Δ10, Δ12), said method comprising introducing into said cell a nucleic acid molecule which encodes a conjugase having an activity of catalyzing the formation of two conjugated double bonds at position 8 and 10 numbered from the carboxyl end of a fatty acyl chain; and a method of producing a cell capable of producing linoleic acids (18:2, Δ9, Δ12), said method comprising introducing into said cell the nucleic acid molecule which encodes a protein having an activity of catalyzing the formation of a double bond at position 12 numbered from the carboxyl end of a fatty acyl.

Other features and advantages of the invention will be apparent from the following detailed description and claims.

### BRIEF DESCRIPTION OF THE FIGURES

FIG. 1 depicts the nucleotide (SEQ ID NO:3) and amino acid (SEQ ID NO:4) sequence of CoFad2 of C. officinalis.

FIG. 2 depicts the nucleotide (SEQ ID NO:1) and protein (SEQ ID NO:2) sequence of CoFac2 of C. officinalis.

FIG. 3 depicts the alignment of the amino acid sequences of CoFac2 (SEQ ID NO:2) and CoFad2 (SEQ ID NO:4) of C. officinalis.

FIG. 4 depicts cluster analysis of CoFac2 with homologous sequences.

FIGS. 5A and 5B depict the results of a Northern blot analysis of CoFac2 and CoFad2.

FIG. 5A depicts an autoradiogram of a Northern blot hybridized with CoFad2 and CoFac2 probes.

FIG. 5B depicts an ethidium bromide gel indicating RNA loading. F, flower buds; L, leaves; S, developing seeds.

FIG. 6 depicts GC analysis of FAMEs from yeast strain Invsc2 expressing CoFac2 and CoFad2 without exogenous substrate

FIG. 7 depicts GC analysis of FAMEs from yeast strain AMY-2α expressing CoFad2 with three exogenous substrates. A: feeding 16:1(9); B: feeding 18:1(9); C: feeding 18:1(9,12).

FIG. 8 depicts GCMS|EI spectra of the MTAD derivatives of novel fatty acids in AMY-2α/pCoFac2 cultures supplemented with 16:1(9Z), (A), 18:1(9Z), (B). The structures assigned to the derivatives are shown with asterisks indicating the original position of the double bonds in the fatty acid. The pairs of peaks with m/z values 236 and 308, in A, 264 and 308, in B, are diagnostic for the loss of R₁ and R₂ fragments, respectively for 16:2(8,10) and 18:2(8,10) derivatives.

FIG. 9 depicts GC analysis of transgenic seeds of Brassica juncea expressing CoFac2. A: the wild type. B: CoFac2 transgenics.

### DETAILED DESCRIPTION OF THE INVENTION

The present invention is based, at least in part, on the discovery of key enzyme coding genes from Calendula. Specifically, the present inventors have identified two homologous genes, CoFad2 (Δ12 desaturase) and CoFac2 (conjugase), of Calendula officinalis. Accordingly, the present invention features methods based on using the presently identified genes to transform plants with the appropriate fatty acid profiles in order to produce conjugated fatty acids.

As used herein, the term “conjugated double bonds” is art recognized and includes conjugated fatty acids (CFAs) containing conjugated double bonds. For example, conjugated double bonds include two double bonds in the relative positions indicated by the formula —CH═CH—CH═CH—. Conjugated double bonds form additive compounds by saturation of the 1 and 4 carbons, so that a double bond is produced between the 2 and 3 carbons.

As used herein, the term “fatty acids” is art recognized and includes a long-chain hydrocarbon based carboxylic acid. Fatty acids are components of many lipids including glycerides. The most common naturally occurring fatty acids are monocarboxylic acids which have an even number of carbon atoms (16 or 18) and which may be saturated or unsaturated. “Unsaturated” fatty acids contain cis double bonds between the carbon atoms. “Polyunsaturated” fatty acids contain more than one double bond and the double bonds are arranged in a methylene interrupted system (—CH═CH—CH₂—CH═CH—). Fatty acids encompassed by the present invention include, for example, linoleic acid, linolenic acid, oleic acid, calendic acid and palmitoleic acid.

Fatty acids are described herein by a numbering system in which the number before the colon indicates the number of carbon atoms in the fatty acid, whereas the number after the colon is the number of double bonds that are present. In the case of unsaturated fatty acids, this is followed by a number in parentheses that indicates the position of the double bonds. Each number in parenthesis is the lower numbered carbon atom of the two connected by the double bond. For example, oleic acid can be described as 18:1(9) and linoleic acid can be described as 18:2(9, 12) indicating 18 carbons, one double bond at carbon 9 and 18 carbons, two double bonds at carbons 9 and 12, respectively.

As used herein, the term “conjugated fatty acids” is art recognized and includes fatty acids containing at least one set of conjugated double bonds. The process of producing conjugated fatty acids is art recognized and includes, for example, a process similar to desaturation, which can result in the introduction of one additional double bond in the existing fatty acid substrate.

As used herein, the term “calendic acid” is art recognized and includes an 18 carbon conjugated fatty acid (8,10,12-18:3). Calendic acid is the major component of the seed oil of Calendula officinalis.

As used herein, the term “linoleic acid” is art recognized and includes an 18 carbon polyunsaturated fatty acid molecule (C₁₇H₂₉COOH) which contains 2 double bonds (18:2(9,12)). The term “Conjugated linoleic acid” (CLA) is a general term for a set of positional and geometric isomers of linoleic acid that possess conjugated double bonds, in the cis or trans configuration. CLA occurs naturally in a wide variety of foods, especially in foods such as cheese that are derived from ruminant animals. CLA is now recognized as a nutritional supplement and an effective inhibitor of epidermal carcinogenesis and for stomach neoplasia in mice, and of carcinogen-induced rat mammary tumors. CLA can prevent adverse effects caused by immune stimulation in chicks, mice and rats, and can decrease the ratio of low density lipoprotein cholesterol (LDL-cholesterol) to high density lipoprotein cholesterol (HDL-cholesterol) in rabbits fed an atherogenic diet. CLA also reduces body fat in mouse, rat and chick models. The effective behavior of CLA in such animal systems suggests similar benefit when provided in the human diet. Linoleic acid can be converted to CLA by chemical methods or by enzymatic isomerization.

As used herein, the term “linolenic acids” is art recognized and includes an 18 carbon polyunsaturated fatty acid molecule (C₁₇H₂₉COOH) that contains three double bonds (18:3(8,10,12)). Linolenic acid occurs as the glyceride in many seed fats and is an essential fatty acid in the diet.

As used herein, the term “oleic acid” is art recognized and includes an 18 carbon monosaturated fatty acid (C₁₇H₃₃COOH) that contains one double bond (18:1,9). Oleic acid is a component of almost all natural fats.

As used herein, the term “conjugase” is art recognized and includes enzymes that are responsible for introducing conjugated double bonds into acyl chains. For example, two conjugases from I. balsamina and M. charantia are able to convert the Δ12 double bond of linoleic acid into two conjugated double bonds at the 11 and 13 positions, resulting in the production of conjugated linolenic acid (18:3(9Z,11E,13E)). In the present invention, for example, expression of CoFac2 in yeast showed that this “conjugase” could convert Δ9 double bonds of 16:1(9Z), 18:1(9Z) and 18:2(9Z,12Z) into two conjugated double bonds at the 8 and 10 positions to produce their corresponding conjugated fatty acids.

As used herein, the term “desaturase” is art recognized and includes enzymes that are responsible for introducing conjugated double bonds into acyl chains. In the present invention, for example, the Δ12 desaturase from Calendula officinalis is a desaturase that can introduce a double bond at position 12 of a fatty acid (e.g., at position 12 of palmitoleic acid (16:1,9) and position 12 of oleic acid (18:1,9)).

Preferred compounds of the invention include CoFad2 and CoFac2. As used herein, the terms CoFad2 (SEQ ID NO 3 and 4) and CoFac2 (SEQ ID NO:1 and 2) refer to two homologous proteins from Calendula officinalis. Both CoFad2 and CoFac2 have sequence similarity to the FAD2 desaturases and related enzymes from plants. CoFAD2 has higher amino acid identity to the FAD2 desaturases (80%), whereas CoFAC2 has approximately equal sequence identity (50%) to both FAD2 desaturases and FAD2-related enzymes including the Δ12 acetylenase of C. alpina, a bifunctional enzyme (oleate 12-hydroxylase: 12-desaturase) of L. fendleri, an epoxygenases from C. palaestina, fatty acid conjugases from C. officinalis, I. balsamina and M. charantia. Expression of CoFad2 cDNA in yeast indicated it encodes a Δ12 desaturase, whereas expression of CoFac2 in yeast revealed that the encoded enzyme was a conjugase which produced conjugated linoleic and linolenic acids from 18:1(9Z) and 18:2(9Z,12Z) substrates, respectively.

Accordingly, one aspect of the present invention features a method of producing a conjugase or a desaturase (e.g., CoFac2 or CoFad2) which includes identifying genes encoding conjugated double bond-forming enzymes in C. officinalis through a PCR-based cloning strategy (Example 2) In a preferred embodiment, CoFac2 and CoFad2 are produced as follows: C. officinalis was grown in a growth chamber at 22° C. with a 16 hour photoperiod at a photon flux density of 150 to 200 μE m^{-2 s}⁻⁵ . The developing seeds at 15 to 30 days after flowering were collected. The embryos were dissected from seeds and used for RNA isolation. The total RNA was isolated from developing embryos according to Qiu and Erickson (1994). The cDNA library was constructed from the total RNA. The first strand cDNA was synthesized by superscript II reverse transcriptase from Gibco-BRL. The second strand cDNA was synthesized by DNA polymerase I from Stratagene. After size fractionation, cDNA inserts larger than 1 kb were ligated into λ Uni-Zap XR vector (Stratagene). The recombinant λ DNAs were then packaged with Gigapack III Gold packaging extract (Stratagene) and plated on NZY plates. The resulting library represented more than 8×10⁶ independent clones. Screening of the cDNA library was performed according to standard methods (Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

For RT-PCR experiments, the single strand cDNA was synthesized by superscript II reverse transcriptase (Gibco-BRL) from total RNA and was then used as the template for PCR reaction. Two degenerate primers (The forward primer: GCXCAC/TGAC/A/GTGC/TGGXCAC/TC/GA and the reverse primer: CATXGTXG/CA/TG/AAAXAG/AG/ATGG/ATG) were designed to target the conserved histidine-rich domains of desaturases. The PCR amplification consisted of 35 cycles with 1 min at 94° C., 1.5 min at 55° C. and 2 min at 72° C. followed by an extension step at 72° C. for 10 min. The amplified products from 400 bp to 600 bp were isolated from agarose gel and purified by a kit (Qiaex II gel purification, Qiagen), and subsequently cloned into the TA cloning vector pCR® 2.1 (Invitrogen). The cloned inserts were then sequenced by PRISM DyeDeoxy Terminator Cycle Sequencing System (Perkin Elmer/Applied Biosystems).

Accordingly, in one aspect, the present invention features a method of producing a conjugase or a desaturase which includes culturing a cell (e.g., a Saccharomyces cerevisae cell) under conditions such that a conjugase or desaturase is produced. The term “overexpressing cell” includes a cell which has been manipulated such that the conjugase or desaturase is overexpressed. The term “overexpressed” or “overexpression” includes expression of a gene product (e.g., CoFac2 having the amino acid sequence of SEQ ID NO:2 encoded by a nucleic acid molecule having the nucleotide sequence of SEQ ID NO:1) at a level greater than that expressed prior to manipulation of the cell or in a comparable cell which has not been manipulated. In one embodiment, the cell can be genetically manipulated (e.g., genetically engineered) to overexpress a level of gene product greater than that expressed prior to manipulation of the cell or in a comparable cell which has not been manipulated. Genetic manipulation can include, but is not limited to, altering or modifying regulatory sequences or sites associated with expression of a particular gene (e.g., by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene routine in the art (including but not limited to use of antisense nucleic acid molecules, for example, to block expression of repressor proteins).

Another aspect of the present invention features a method of modulating the production of fatty acids comprising culturing cells transformed by the nucleic acid molecules of the present invention (e.g., a conjugase or a desaturase) such that modulation of fatty acid production occurs (e.g., production of conjugated linoleic acid is enhanced). The method of culturing cells transformed by the nucleic acid molecules of the present invention (e.g., CoFac2 and CoFad2) to modulate the production of fatty acids is referred to herein as “biotransformation.” The biotransformation processes can utilize recombinant cells and/or conjugases and desaturases described herein. The term “biotransformation process”, also referred to herein as “bioconversion processes”, includes biological processes which results in the production (e.g., transformation or conversion) of any compound (e.g., substrate, intermediate or product) which is upstream of a fatty acid conjugase or desaturase to a compound (e.g., substrate, intermediate or product) which is downstream of said fatty acid conjugase or desaturase, in particular, a conjugated fatty acid. In one embodiment, the invention features a biotransformation process for the production of a conjugated fatty acid comprising contacting a cell which overexpresses at least one fatty acid conjugase or desaturase with at least one appropriate substrate under conditions such that said conjugated fatty acid is produced and recovering said fatty acid. In a preferred embodiment, the invention features a biotransformation process for the production of fatty acids comprising contacting a cell which overexpresses CoFac2 with an appropriate substrate (e.g., oleic acid and linoleic acid) under conditions such that conjugated linoleic acid (CLA) is produced and recovering said conjugated linoleic acid (CLA). Conditions under which conjugated linoleic acid (CLA) is produced can include any conditions which result in the desired production of conjugated linoleic acid (CLA). In another preferred embodiment, the invention features a biotransformation process for the production of linolenic acid comprising contacting a cell which overexpresses CoFad2 with appropriate substrates (e.g., oleic acid) under conditions such that conjugated linoleic acid (CLA) is produced and recovering said conjugated linoleic acid (CLA). Conditions under which conjugated linoleic acid (CLA) is produced can include any conditions which result in the desired production of conjugated linoleic acid (CLA).

The cell(s) and/or enzymes used in the biotransformation reactions are in a form allowing them to perform their intended function (e.g., producing a desired fatty acids). The cells can be whole cells, or can be only those portions of the cells necessary to obtain the desired end result. The cells can be suspended (e.g., in an appropriate solution such as buffered solutions or media), rinsed (e.g., rinsed free of media from culturing the cell), acetone-dried, immobilized (e.g., with polyacrylamide gel or k-carrageenan or on synthetic supports, for example, beads, matrices and the like), fixed, cross-linked or permeablized (e.g., have permeablized membranes and/or walls such that compounds, for example, substrates, intermediates or products can more easily pass through said membrane or wall). The type of cell can be any cell capable of being used within the methods of the invention, e.g., plant, bacterial or yeast cells.

Purified or unpurified fatty acid conjugases or desaturases are also to be used in the biotransformation reactions. The enzyme can be in a form that allows it to perform its intended function (e.g., obtaining the desired conjugated fatty acid). For example, the enzyme can be in free form or immobilized. Purified or unpurified fatty acid conjugase or desaturase can be contacted in one or more in vitro reactions with appropriate substrate(s) such that the desired product is produced. Art-recognized techniques can be used to prepare the cells and/or enzymes including those described in Hikichi et al., U.S. Pat. No. 5,518,906, Yamashita et al, U.S. Pat. No. 5,089,276, Moriya et al., U.S. Pat. No. 5,932,457, Warnek et al., U.S. Pat. No. 5,912,164, Chen et al., U.S. Pat. No. 5,756,536, Debono et al., U.S. Pat. No. 5,534,420, Van Solingen., U.S. Pat. No. 5,856,165, Perkins et al., U.S. Pat. No. 5,925,538, Hikichi et al., Haynie et al., U.S. Pat. No. 5,599,689 and European Pat. Application No. EP 590857.

In another embodiment, the cell can be physically or environmentally manipulated to overexpress a level of gene product greater than that expressed prior to manipulation of the cell or in a comparable cell which has not been manipulated. For example, a cell can be treated with or cultured in the presence of an agent known or suspected to increase transcription of a particular gene and/or translation of a particular gene product such that transcription and/or translation are enhanced or increased.

In a preferred embodiment, a cell overexpresses conjugase or desaturase which is “plant derived.” The term “plant-derived” or “derived-from”, for example a plant, includes a gene product (e.g., the CoFad2 or CoFac2 gene product) which is encoded by a plant gene. In a preferred embodiment, the gene product is derived from Calendula (e.g., is Calendula-derived). The term “derived from Calendula” or “Calendula-derived” includes a gene product (e.g., e.g., the CoFad2 or CoFac2 gene product) which is encoded by a Calendula gene. In a particularly preferred embodiment, the gene product (e.g., CoFac2 or CoFad2) is derived from Calendula officinalis (e.g., is Calendula officinalis-derived). Included within the scope of the present invention are plant-derived gene products and/or Calendula-derived gene products (e.g., C. officinalis derived gene products) that are encoded by naturally-occurring plant and/or Calendula genes (e.g., C. officinalis genes), for example, the genes identified by the present inventors. Further included within the scope of the present invention are plant-derived gene products and/or Calendula-derived gene products (e.g., C. officinalis-derived gene products) that are encoded by plant and/or Calendula genes (e.g., C. officinalis-genes) which differ from naturally-occurring plant and/or Calendula genes (e.g., C. officinalis-genes), for example, genes which have nucleic acids that are mutated, inserted or deleted, but which encode proteins substantially similar to the naturally-occurring gene products of the present invention. For example, it is well understood that one of skill in the art can mutate (e.g., substitute) nucleic acids which, due to the degeneracy of the genetic code, encode for an identical amino acid as that encoded by the naturally-occurring gene. Moreover, it is well understood that one of skill in the art can mutate (e.g., substitute) nucleic acids which encode for conservative amino acid substitutions. It is further well understood that one of skill in the art can substitute, add or delete amino acids to a certain degree without substantially affecting the function of a gene product as compared with a naturally-occurring gene product, each instance of which is intended to be included within the scope of the present invention.

The term “culturing” includes maintaining and/or growing a living cell of the present invention (e.g., maintaining and/or growing a culture or strain) such that it can perform its intended function. In one embodiment, a cell of the invention is cultured in liquid media. In another embodiment, a cell of the invention is cultured in solid media or semi-solid media. In a preferred embodiment, a cell of the invention is cultured in media (e.g., a sterile, liquid media) comprising nutrients essential or beneficial to the maintenance and/or growth of the cell (e.g., carbon sources or carbon substrate, for example carbohydrate, hydrocarbons, oils, fats, fatty acids, organic acids, and alcohol's; nitrogen sources, for example, peptone, yeast extracts, meat extracts, malt extracts, urea, ammonium sulfate, ammonium chloride, ammonium nitrate and ammonium phosphate; phosphorus sources, for example, monopotassiun phosphate or dipotassium phosphate; trace elements (e.g., metal salts), for example magnesium salts (e.g., magnesium sulfate), cobalt salts and/or manganese salts; as well as growth factors such as amino acids, vitamins, growth promoters, and the like).

Preferably, cells of the present invention are cultured under controlled pH. The term “controlled pH” includes any pH which results in production of the desired product (e.g., a conjugase). In one embodiment cells are cultured at a pH of about 7. In another embodiment, cells are cultured at a pH of between 6.0 and 8.5. The desired pH may be maintained by any number of methods known to those skilled in the art.

Also preferably, cells of the present invention are cultured under controlled aeration. The term “controlled aeration” includes sufficient aeration (e.g., oxygen) to result in production of the desired product (e.g., a fatty acid conjugase). In one embodiment, aeration is controlled by regulating oxygen levels in the culture, for example, by regulating the amount of oxygen dissolved in culture media. Preferably, aeration of the culture is controlled by agitating the culture. Agitation may be provided by a propeller or similar mechanical agitation equipment, by revolving or shaking the fermentor or by various pumping equipment. Aeration may be further controlled by the passage of sterile air through the medium (e.g., through the fermentation mixture). Also preferably, cells of the present invention are cultured without excess foaming (e.g., via addition of antifoaming agents).

Moreover, cells of the present invention can be cultured under controlled temperatures. The term “controlled temperature” include any temperature which results in production of the desired product (e.g., a conjugated fatty acid). In one embodiment, controlled temperatures include temperatures between 15° C. and 95° C. In another embodiment, controlled temperatures include temperatures between 15° C. and 70° C. Preferred temperatures are between 20° C. and 55° C., more preferably between 30° C. and 45° C.

Cells can be cultured (e.g., maintained and/or grown) in liquid media and preferably are cultured, either continuously or intermittently, by conventional culturing methods such as standing culture, test tube culture, shaking culture (e.g, rotary shaking culture, shake flask culture, etc.), aeration spinner culture, or fermentation. In a preferred embodiment, the cells are cultured in shake flasks. In a more preferred embodiment, the cells are cultured in a fermentor (e.g., a fermentation process). Fermentation processes of the present invention include, but are not limited to, batch. fed-batch and continuous processes or methods of fermentation. The phrase “batch process” or “batch fermentation” refers to a closed system in which the composition of media, nutrients, supplemental additives and the like is set at the beginning of the fermentation and not subject to alteration during the fermentation, however, attempts may be made to control such factors as pH and oxygen concentration to prevent excess media acidification and/or cell death. The phrase “fed-batch process” or “fed-batch” fermentation refers to a batch fermentation with the exception that one or more substrates or supplements are added (e.g., added in increments or continuously) as the fermentation progresses. The phrase “continuous process” or “continuous fermentation” refers to an open system in which a defined fermentation media is added continuously to a fermentor and an equal amount of used or “conditioned” media is simultaneously removed, preferably for recovery of the desired product (e.g., conjugated fatty acid). A variety of such processes have been developed and are well-known in the art.

The phrase “culturing under conditions such that conjugated fatty acid is produced” includes maintaining and/or growing cells under conditions (e.g., temperature, pressure, pH, duration, etc.) appropriate or sufficient for obtaining production of a particular conjugated fatty acid or for obtaining desired yields of the particular conjugated fatty acid being produced. For example, culturing is continued for a time sufficient to produce the desired amount of conjugated fatty acid. Preferably, culturing is continued for a time sufficient to substantially reach maximal production of conjugated fatty acid. In one embodiment, culturing is continued for about 12 to 24 hours. In another embodiment, culturing is continued for about 24 to 36 hours, 36 to 48 hours, 48 to 72 hours, 72 to 96 hours, 96 to 120 hours, or greater than 120 hours.

In producing conjugated fatty acids, it may further be desirable to culture cells of the present invention in the presence of supplemental fatty acid biosynthetic substrates. The term “supplemental fatty acid biosynthetic substrate” includes an agent or compound which, when brought into contact with a cell or included in the culture medium of a cell, serves to enhance or increase conjugated fatty acid biosynthesis. Supplemental fatty acid biosynthetic substrates of the present invention can be added in the form of a concentrated solution or suspension (e.g., in a suitable solvent such as water or buffer) or in the form of a solid (e.g., in the form of a powder). Moreover, supplemental fatty acid biosynthetic substrates of the present invention can be added as a single aliquot, continuously or intermittently over a given period of time.

The methodology of the present invention can further include a step of recovering the conjugated fatty acid. The term “recovering” the conjugated fatty acid includes extracting, harvesting, isolating or purifying the conjugated fatty acid from culture media. Recovering the conjugated fatty acid can be performed according to any conventional isolation or purification methodology known in the art including, but not limited to, treatment with a conventional resin (e.g., anion or cation exchange resin, non-ionic adsorption resin, etc.), treatment with a conventional adsorbant (e.g., activated charcoal, silicic acid, silica gel, cellulose, alumina, etc.), alteration or pH, solvent extraction (e.g., with a conventional solvent such as alcohol and the like), dialysis, filtration, concentration, crystallization, recrystallization, pH adjustment, lyophilization and the like. For example, a conjugated fatty acid (e.g., CLA) can be recovered from culture media by first removing the cells from the culture. Media is then passed through or over a cation exchange resin to remove cations and then through or over an anion exchange resin to remove inorganic anions and organic acids having stronger acidities than the conjugated fatty acid of interest (e.g., CLA).

Preferably, a conjugated fatty acid is “extracted”, “isolated” or “purified” such that the resulting preparation is substantially free of other media components. The language “substantially free of other media components” includes preparations of conjugated fatty acid in which the compound is separated from media components of the culture from which it is produced. In one embodiment, the preparation has greater than about 80% (by dry weight) of conjugated fatty acid (e.g., less than about 20% of other media components), more preferably greater than about 90% of conjugated fatty acid (e.g., less than about 10% of other media components), still more preferably greater than about 95% of conjugated fatty acid (e.g., less than about 5% of other media components), and most preferably greater than about 98-99% conjugated fatty acid (e.g., less than about 1-2% other media components. When the conjugated fatty acid is derivatized to a salt (e.g. a calendic acid salt), the conjugated fatty acid is preferably further free of chemical contaminants associated with the formation of the salt. When the conjugated fatty acid is derivatized to an alcohol, the conjugated fatty acid is preferably further free of chemical contaminants associated with the formation of the alcohol.

Ranges intermediate to the above-recited values, e.g., at 80-85% (by dry weight) or 75-80% (by dry weight) of conjugated fatty acids are also intended to be encompassed by the present invention. Values and ranges included and/or intermediate within the ranges set forth herein are also intended to be within the scope of the present invention. For example, conjugated fatty acid preparations of 81, 82, 83, 84, 85, 86, 87, 88 and 89 percent (by dry weight) are intended to be included within the range of 80 to 90 percent (by dry weight).

The present invention further features recombinant vectors that include nucleic acid sequences that encode plant gene products as described herein, preferably Calendula gene products, more preferably Calendula offcinalis gene products, even more preferably Calendula officinalis CoFac2 and CoFad2 gene products. The term recombinant vector includes a vector (e.g., plasmid) that has been altered, modified or engineered such that it contains greater, fewer or different nucleic acid sequences than those included in the native vector or plasmid. In one embodiment, a recombinant vector includes the nucleic acid sequence encoding at least one Calendula offcinalis fatty acid conjugase enzyme operably linked to regulatory sequences. The phrase operably linked to regulatory sequence(s)” means that the nucleotide sequence of interest is linked to the regulatory sequence(s) in a manner which allows for expression (e.g., enhanced, increased, constitutive, basal, attenuated, decreased or repressed expression) of the nucleotide sequence, preferably expression of a gene product encoded by the nucleotide sequence (e.g., when the recombinant vector is introduced into a cell). Exemplary vectors are described in further detail herein as well as in, for example, Frascotti et al., U.S. Pat. No. 5,721,137, the contents of which are incorporated herein by reference.

The term “regulatory sequence” includes nucleic acid sequences which affect (e.g., modulate or regulate) expression of other (non-regulatory) nucleic acid sequences. In one embodiment, a regulatory sequence is included in a recombinant vector in a similar or identical position and/or orientation relative to a particular gene of interest as is observed for the regulatory sequence and gene of interest as it appears in nature, e.g., in a native position and/or orientation. For example, a gene of interest (e.g., Calendula offcinalis CoFac2 or CoFad2 gene) can be included in a recombinant vector operably linked to a regulatory sequence which accompanies or is adjacent to the Calendula offcinalis CoFac2 or CoFad2 gene in the natural organism (e.g., operably linked to “native” CoFac2 or CoFad2 regulatory sequence (e.g., to the “native” CoFac2 or CoFad2 promoter). Alternatively, a gene of interest (e.g., a Calendula offcinalis CoFac2 or CoFad2 gene) can be included in a recombinant vector operably linked to a regulatory sequence which accompanies or is adjacent to another (e.g., a different) gene in the natural organism. For example, a Calendula offcinalis CoFac2 or CoFad2 gene can be included in a vector operably linked to non-CoFac2 or non-CoFad2 regulatory sequences from Calendula officinalis. Alternatively, a gene of interest (e.g., Calendula officinalis CoFac2 or CoFad2 gene) can be included in a vector operably linked to a regulatory sequence from another organism. For example, regulatory sequences from other microbes (e.g., other bacterial regulatory sequences, bacteriophage regulatory sequences and the like) can be operably linked to a particular gene of interest.

Preferred regulatory sequences include promoters, enhancers, termination signals and other expression control elements (e.g., binding sites for transcriptional and/or translational regulatory proteins, for example, in the transcribed mRNA). Such regulatory sequences are described, for example, in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. Regulatory sequences include those which direct constitutive expression of a nucleotide sequence in a cell (e.g., constitutive promoters and strong constitutive promoters), those which direct inducible expression of a nucleotide sequence in a cell (e.g., inducible promoters, for example, xylose inducible promoters) and those which attenuate or repress expression of a nucleotide sequence in a cell (e.g., attenuation signals or repressor sequences). It is also within the scope of the present invention to regulate expression of a gene of interest by removing or deleting regulatory sequences. For example, sequences involved in the negative regulation of transcription can be removed such that expression of a gene of interest is enhanced.

In one embodiment, a recombinant vector of the present invention includes nucleic acid sequences that encode at least one plant gene product (e.g., CoFac2 or CoFad2) operably linked to a promoter or promoter sequence. Preferred promoters of the present invention include Calendula promoters.

In yet another embodiment, a recombinant vector of the present invention includes a terminator sequence or terminator sequences (e.g., transcription terminator sequences). The term “terminator sequences” includes regulatory sequences which serve to terminate transcription of mRNA. Terminator sequences (or tandem transcription terminators) can further serve to stabilize mRNA (e.g., by adding structure to mRNA), for example, against nucleases.

In yet another embodiment, a recombinant vector of the present invention includes antibiotic resistance sequences. The term “antibiotic resistance sequences” includes sequences which promote or confer resistance to antibiotics on the host organism (e.g., Calendula). In one embodiment, the antibiotic resistance sequences are selected from the group consisting of cat (chloramphenicol resistance), tet (tetracycline resistance) sequences, erm (erythromycin resistance) sequences, neo (neomycin resistance) sequences and spec (spectinomycin resistance) sequences. Recombinant vectors of the present invention can further include homologous recombination sequences (e.g., sequences designed to allow recombination of the gene of interest into the chromosome of the host organism). For example, amyE sequences can be used as homology targets for recombination into the host chromosome.

It will further be appreciated by one of skill in the art that the design of a vector can be tailored depending on such factors as the choice of cell to be genetically engineered, the level of expression of gene product desired and the like.

Another aspect of the present invention features isolated nucleic acid molecules that encode Calendula proteins (e.g., C. officinalis) proteins, for example, Calendula cojugase (e.g., CoFac2) or Calendula Δ12 desaturase (e.g., CoFad2)). The term “nucleic acid molecule” includes DNA molecules (e.g., cDNA or chromosomal DNA) and RNA molecules (e.g., mRNA) and analogs of the DNA or RNA generated using nucleotide analogs. The nucleic acid molecule can be single-stranded or double-stranded, but preferably is double-stranded DNA. Preferably, an “isolated” nucleic acid molecule is free of sequences which naturally flank the nucleic acid molecule (i.e., sequences located at the 5′ and 3′ ends of the nucleic acid molecule) in the chromosomal DNA of the organism from which the nucleic acid is derived (e.g., is free of naturally-occurring regulatory sequences). In various embodiments, an isolated nucleic acid molecule can contain less than about 5 kb, 4 kb, 3 kb, 2 kb, 1 kb, 0.5 kb or 0.1 kb of nucleotide sequences which naturally flank the nucleic acid molecule in chromosomal DNA of the cell from which the nucleic acid molecule is derived. Moreover, an “isolated” nucleic acid molecule, such as a cDNA molecule, can be substantially free of other cellular materials when produced by recombinant techniques, or substantially free of chemical precursors or other chemicals when chemically synthesized.

A nucleic acid molecule of the present invention (e.g., a nucleic acid molecule having the nucleotide sequence of SEQ ID NO:1 or SEQ ID NO:3, can be isolated using standard molecular biology techniques and the sequence information provided herein. For example, nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989) or can be isolated by the polymerase chain reaction using synthetic oligonucleotide primers designed based upon the sequence SEQ ID NO:1 or SEQ ID NO:3. A nucleic acid of the invention can be amplified using cDNA, mRNA or alternatively, genomic DNA, as a template and appropriate oligonucleotide primers according to standard PCR amplification techniques. In another preferred embodiment, an isolated nucleic acid molecule of the invention comprises a nucleic acid molecule which is a complement of the nucleotide sequence shown in SEQ ID NO:1 or SEQ ID NO:3.

In a preferred embodiment, an isolated nucleic acid molecule comprises at least one of the nucleotide sequences set forth as SEQ ID NO:1 or SEQ ID NO:3. In another preferred embodiment, an isolated nucleic acid molecule comprises at least two, three or four of the nucleotide sequences set forth as SEQ ID NO:1 or SEQ ID NO:3. For example, a preferred isolated nucleic acid molecule of the present invention can include the nucleotide sequences of SEQ ID NO:1 and SEQ ID NO:3, preferably linked such that the proteins encoded by the nucleotide sequences of SEQ ID NO:1 and SEQ ID NO:3 are each produced when the isolated nucleic acid molecule is expressed in a cell.

In still another preferred embodiment, an isolated nucleic acid molecule of the present invention comprises a nucleotide sequence which is at least about 60-65%, preferably at least about 70-75%, more preferable at least about 80-85%, and even more preferably at least about 90-95% or more identical to a nucleotide sequence set forth as SEQ ID NO:1 or SEQ ID NO:3. In another embodiment, an isolated nucleic acid molecule hybridizes under stringent conditions to a nucleic acid molecule having a nucleotide sequence set forth as SEQ ID NO:1 or SEQ ID NO:3. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2× SSC, 0.1% SDS at 50-65° C. Preferably, an isolated nucleic acid molecule of the invention that hybridizes under stringent conditions to the sequence of SEQ ID NO:1 or SEQ ID NO:3, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

Ranges intermediate to the above-recited values, e.g., isolated nucleic acid molecules comprising a nucleotide sequence which is about 20-60%, 65-70%, 75-80% or 85-90% identical to the nucleotide sequence set forth in SEQ ID NO:1 or SEQ ID NO:3 are also intended to be encompassed by the present invention. Values and ranges included and/or intermediate within the ranges set forth herein are also intended to be within the scope of the present invention. For example, isolated nucleic acid molecules comprising a nucleotide sequence which is about 81%, 82%, 83%, and 84% identical to the nucleotide sequence set forth in SEQ ID NO:1 or SEQ ID NO:3 are intended to be included within the range of 80-85% identical to the nucleotide sequence set forth in SEQ ID NO:1 or SEQ ID NO:3.

Another aspect of the present invention features isolated proteins (e.g., isolated CoFac2 and CoFad2 proteins). In one embodiment, proteins (e.g., isolated CoFac2 and CoFad2 proteins) are produced by recombinant DNA techniques and can be isolated from cells of the present invention by an appropriate purification scheme using standard protein purification techniques. In another embodiment, proteins (e.g., CoFac2 and CoFad2 proteins) are synthesized chemically using standard peptide synthesis techniques.

An “isolated” or “purified” protein (e.g. isolated CoFac2 and CoFad2 proteins). is substantially free of cellular material or other contaminating proteins from the cell from which the protein is derived, or substantially free from chemical precursors or other chemicals when chemically synthesized. In one embodiment, an isolated or purified protein has less than about 30% (by dry weight) of contaminating protein or chemicals, more preferably less than about 20% of contaminating protein or chemicals, still more preferably less than about 10% of contaminating protein or chemicals, and most preferably less than about 5% contaminating protein or chemicals.

In a preferred embodiment, an isolated protein of the present invention (e.g., isolated CoFac2 and CoFad2 proteins) has an amino acid sequence shown in SEQ ID NO:2 or SEQ ID NO:4. In other embodiments, an isolated protein of the present invention comprises an amino acid sequence at least about 60% identical, preferably about 70% identical, more preferably about 80% identical, even more preferably about 90% identical and even more preferably about 95% identical to the amino acid sequence of SEQ ID NO:2 or SEQ ID NO:4. To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions×100), preferably taking into account the number of gaps and size of said gaps necessary to produce an optimal alignment.

Ranges intermediate to the above-recited values, e.g., isolated proteins comprising an amino acid sequence which is about 20-60%, 60-70%, 70-80% or 80-90% identical to the amino acid sequence set forth in SEQ ID NO:2 or SEQ ID NO:4 are also intended to be encompassed by the present invention. Values and ranges included and/or intermediate within the ranges set forth herein are also intended to be within the scope of the present invention. For example, isolated proteins comprising an amino acid sequence which is about 91%, 92%, 93%, 94%, 95%, 96%, 97%, 98%, and 99% identical to the amino acid sequence set forth in SEQ ID NO:2 or SEQ ID NO:4 are intended to be included within the range of about 90% identical to the amino acid sequence set forth in SEQ ID NO:2 or SEQ ID NO:4.

The comparison of sequences and determination of percent homology between two sequences can be accomplished using a mathematical algorithm. A preferred, non-limiting example of a mathematical algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul et al. (1990) J. Mol. Biol. 215:403-10. BLAST nucleotide searches can be performed with the NBLAST program, score 100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (1997) Nucleic Acids Research 25(17):3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

In another preferred embodiment, the percent homology between two amino acid sequences can be accomplished using the GAP program in the GCG software package (available at http://www.gcg.com), using either a Blossom 62 matrix or a PAM250 matrix, and a gap weight of 12, 10, 8, 6, or 4 and a length weight of 2, 3, or 4. In yet another preferred embodiment, the percent homology between two nucleic acid sequences can be accomplished using the GAP program in the GCG software package (available at http://www.gcg.com), using a gap weight of 50 and a length weight of 3.

This invention is further illustrated by the following examples which should not be construed as limiting. The contents of all references, patents and published patent applications cited throughout this application are incorporated herein by reference.

### EXAMPLES

General Methodology:

Plant Materials:

C. officinalis was grown in a growth chamber at 22° C. with a 16 hour photoperiod at a photon flux density of 150 to 200 μE m⁻² s^{31 1}. The developing seeds at 15 to 30 days after flowering were collected. The embryos were dissected from seeds and used for RNA isolation.

Construction and Screening of cDNA Library:

The total RNA was isolated from developing embryos according to Qiu et al. (1994) Plant Mol. Biol. Reporter 12:209-214. The cDNA library was constructed from the total RNA. The first strand cDNA was synthesized by superscript II reverse transcriptase from Gibco-BRL. The second strand cDNA was synthesized by DNA polymerase I from Stratagene. After size fractionation, cDNA inserts larger than 1 kb were ligated into λ Uni-Zap XR vector (Stratagene). The recombinant λ DNAs were then packaged with Gigapack III Gold packaging extract (Stratagene) and plated on NZY plates. The resulting library represented more than 8×10⁶ independent clones. Screening of the cDNA library was performed according to standard methods (Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

RT-PCR:

For RT-PCR experiments, the single strand cDNA was synthesized by superscript II reverse transcriptase (Gibco-BRL) from total RNA and was then used as the template for PCR reaction. Two degenerate primers (The forward primer: GCXCAC/TGAC/A/GTGC/TGGXCAC/TC/GA and the reverse primer: CATXGTXG/CA/TG/AAAXAG/AG/ATGG/ATG) were designed to target the conserved histidine-rich domains of desaturases. The PCR amplification consisted of 35 cycles with 1 min at 94° C., 1.5 min at 55° C. and 2 min at 72° C. followed by an extension step at 72° C. for 10 min. The amplified products from 400 bp to 600 bp were isolated from agarose gel and purified by a kit (Qiaex II gel purification, Qiagen), and subsequently cloned into the TA cloning vector pCR® 2.1 (Invitrogen). The cloned inserts were then sequenced by PRISM DyeDeoxy Terminator Cycle Sequencing System (Perkin Elmer/Applied Biosystems).

Phylogenetic Analysis:

For phylogenetic analysis, predicted amino acid sequences were aligned using CLUSTALW (v1.60) (Thompson et al. (1998) Nucl. Acids Res. 22;4673-4680) with the default parameters, including gap open and extension penalties of 10 and 0.05 respectively for both pairwise and multiple alignments. The BLOSUM 30 protein weight matrix was used for pairwise alignments and the BLOSUM series for multiple alignments. CLUSTALW was used to determine dendrograms representing a neighbor-joining analysis of sequence distances. Bootstrap analysis was performed with 1000 iterations and visualized with the TreeView program (Page, 1996).

Northern Blot Analysis:

For northern blot analysis, 7 μg total RNAs isolated from flower buds, leaves and developing seeds of C. officinalis as described above were fractionated in a formaldehyde-agarose gel. After electrophoresis, RNAs were transferred to Hybond membrane (Amersham Pharmacia) using 10× SSC transferring solution and were then fixed to the membrane by UV crosslinking. Filter-bound RNAs were then hybridized with the radiolabelled cDNA probes at 68° C. for 1 h in Quickhyb (Stratagene). After hybridization, the blots were washed once at room temperature for half an hour with a solution of 2× SSC and 1% SDS, and once at 65° C. for half an hour with a solution of 0.1× SSC and 0.1% SDS.

Expression of CoFad2 and CoFac2 in Yeast (Saccharomyces cerevisiae):

The open reading frames of CoFad2 and CoFac2 were amplified by PCR using the Precision Plus enzyme (Stratagene) and cloned into a TA cloning vector (pCR® 2.1, Invitrogen). Having confirmed that the PCR products were identical to the original cDNAs by sequencing, the fragments were then released by a BamHI-EcoRI double digestion and inserted into the yeast expression vector pYES2 (Invitrogen) under the control of the inducible promoter GAL1.

Yeast strains InvSc2 (Invitrogen) and AMY-2α (the genotype: MATα; CYTb5, ole1(ΔBstEII)::LEU2, trp1-1, can1-100, ura3-1, ade2-1, HIS3) (Mitchell et al. (1995) J. Biol. Chem. 270:29766-29772) were transformed with the expression constructs using the lithium acetate method and transformants were selected on minimal medium plates lacking uracil (Gietz et al. (1992) Nucleic acids Res. 20:1425; Covello et al. (1996) Plant. Physiol. 1111:223-226).

Transformants were first grown in minimal medium lacking uracil and containing glucose (CM-ura, Ausubel et al. (1995) Current Protocols in Molecular Biology, John Wiley & Sons, New York) at 28° C. After overnight culture, the cells were spun down, washed and resuspended in distilled water. Minimal medium with 2% galactose replacing glucose, and with or without 0.3 mM substrate fatty acids in the presence of 0.1% Tergitol, was inoculated with the yeast transformant cell suspension and incubated at 20° C. for three days followed by 15° C. for three days. For the AMY2α strain, media were supplemented with 0.3 mM 17:1(10Z) and 0.1% Tergitol.

Fatty Acid Analysis:

Yeast cultures were pelleted by centrifugation (4000 g , 10 min.) and pellets were washed with 10 mL 1% Tergitol solution and 2×10 mL H₂O. The yeast pellet was dried under vacuum at ambient temperature. To the dried pellet in a glass culture tube was added 1 mL methanol and the pellet was dispersed using a high speed homogenizer. To this mixture was added 2 mL 0.5M sodium methoxide in methanol. The tube was flushed with nitrogen, sealed and heated to 50° C. for 1 hour. The cooled mixture was extracted with 2×2 mL hexane. The pooled hexane was washed with 2 mL H₂O and concentrated under N₂ for GC or GC/MS analysis.

Fatty acid methyl ester (FAME) analysis was carried out using a Hewlett Packard 6890 series gas chromatograph equipped with a DB-23 fused silica column (30 m×0.25 mm i.d., 0.25 μm film thickness; J&W Scientific, Fulsom, Calif.) with a temperature program of 180° C. for 1 min, 4° C./min to 240° C., hold for 15 min.

For conjugated polyene analysis, FAME were derivatized with 4-methyl-1,2,4-triazoline-3,5-dione (MTAD) (Dobson G. (1998) JACOS 75(2):137-142). 100 μl of a dilute solution of MTAD (<1 mg/mL, slight pink color) in CHCl₃ at 0° C. was added to dry FAME from yeast cells with agitation for 5 to 10 seconds. A dilute solution of 1,3-hexadiene (excess) was then added to neutralize reactants (removal of color). The tube was dried under nitrogen and the residue re-dissolved in CHCl₃.

GC/MS analysis was performed in standard El mode using a Fisons VG TRIO 2000 mass spectrometer (VG Analytical, UK) controlled by Masslynx version 2.0 software, coupled to a GC 8000 Series gas chromatograph. For FAME analysis, a DB-23 column was used with the temperature program described above. For MTAD derivative analysis, a DB-5 column (60M×0.32 mm i.d., 0.25 μm film thickness, J&W Scientific, Folsom, Calif.) that was temperature-programmed at 50° C. for 1 min, increased at 20° C./min to 160° C., then 5° C./min to 350° C. and held for 15 min.

Transformation of Brassica juncea:

The hypocotyls of 5-6 day seedlings of B. juncea were used as explants for inoculation with the Agrobacterium tumefaciens that hosts a binary vector with the full-length CoFac2 cDNA under the control of the napin promoter. The subsequent co-cultivation and regeneration were essentially according to Radke et al. (1992) Plant Cell Reports 11:499-505.

### Example 1

### Linoleic Acid is the Precursor of Calendic Acid

Calendula officinalis is an annual flowering plant that can accumulate a significant amount of calendic acid in its seeds. In order to obtain the molecular information underlying the biosynthesis of calendic acid, the fatty acid profiles of the seeds, leaves and flowering buds of Calendula officinalis was analyzed.

Table 1 is the fatty acid composition of lipids isolated from full-expanded leaves, unopened flower buds and mature seeds. Calendic acid is a major fatty acid of the lipids in the seeds that accounts for more than 46% of the total fatty acids. Following calendic acid is linoleic acid, which comprises approximately 34% of the total fatty acids in the seeds. Oleic acid accounts only for 4%. A trace amount of CLA (8,10-18:2) was also found in the seeds. Calendic acid was not present in either leaves or flower buds. However, linolenic acid is a major fatty acid that accounts for 43% of the total fatty acids in leaves. Palmitic and linoleic acids account for 15% and 13% of the total fatty acids in leaves, respectively. In flower buds, linoleic acids are the most abundant lipid fatty acids, then linolenic (26%) and palmitic (17%) acids. Thus, linolenic acid is preferentially accumulated in leave and flower buds whereas in seeds it is less than one percent of total fatty acids. Palmitoleic acid is not present in any part (seeds, leaves, flower buds) of Calendula officinalis.

The results of the fatty acid profiles of the seeds, leaves and flowering buds of Calendula officinalis suggest that linoleic acid may be the immediate precursor for biosynthesis of calendic acid.

TABLE 1
Fatty acid profiles of Calendula officinalis
(weight percentage)

| Fatty acids | Flower buds | Leaves | Seeds |
|---|---|---|---|
| 16:0 | 17.66 | 15.41 | 3.76 |
| 16:1(9) | 0 | 0 | 0 |
| 18:0 | 2.23 | 0.62 | 1.69 |
| 18:1(9) | 1.44 | 0.63 | 4.04 |
| 18:2(9, 12) | 33.48 | 13.87 | 34.04 |
| 18:2(8, 10) | 0 | 0 | 0.27 |
| 18:3(9, 12, 15) | 26.49 | 43.87 | 0.95 |
| 18:3(8, 10, 12) | 0 | 0 | 46.28 |

### Example 2

### Identification of CoFac2 and CoFad2

To identify genes encoding conjugated double bond-forming enzymes in C. officinalis, a PCR-based cloning strategy was adopted. Sequencing of PCR products revealed three types of inserts related to desaturases. One had high sequence similarity to ω-3 desaturases (FAD3). The other two shared amino acid sequence similarity to various Δ12 desaturases (FAD2) and related enzymes, such as an acetylenase from Crepis alpina.

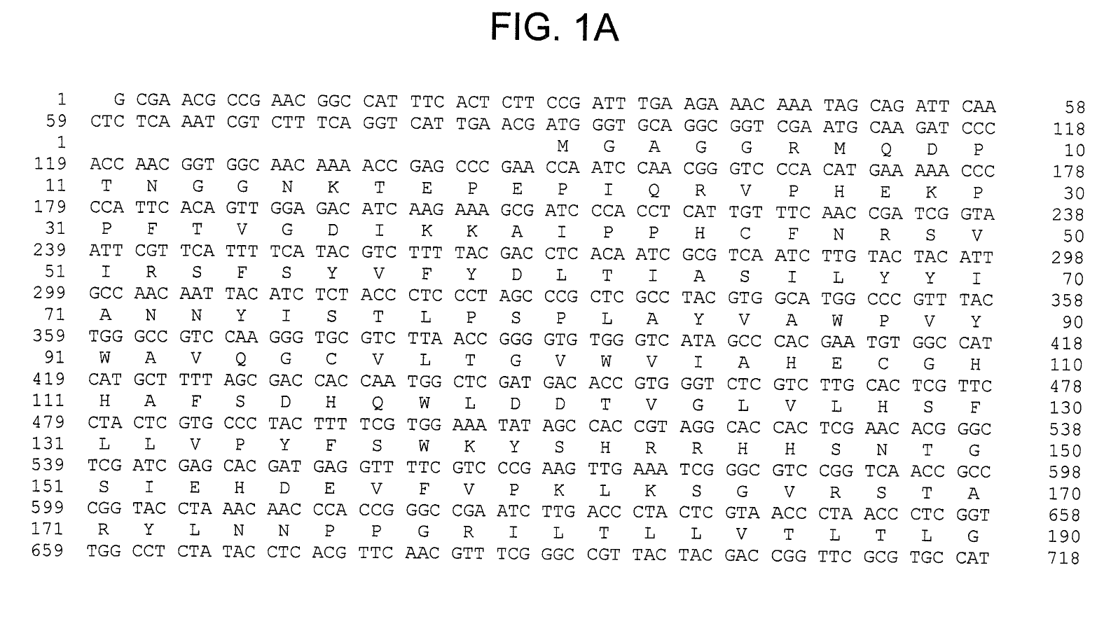

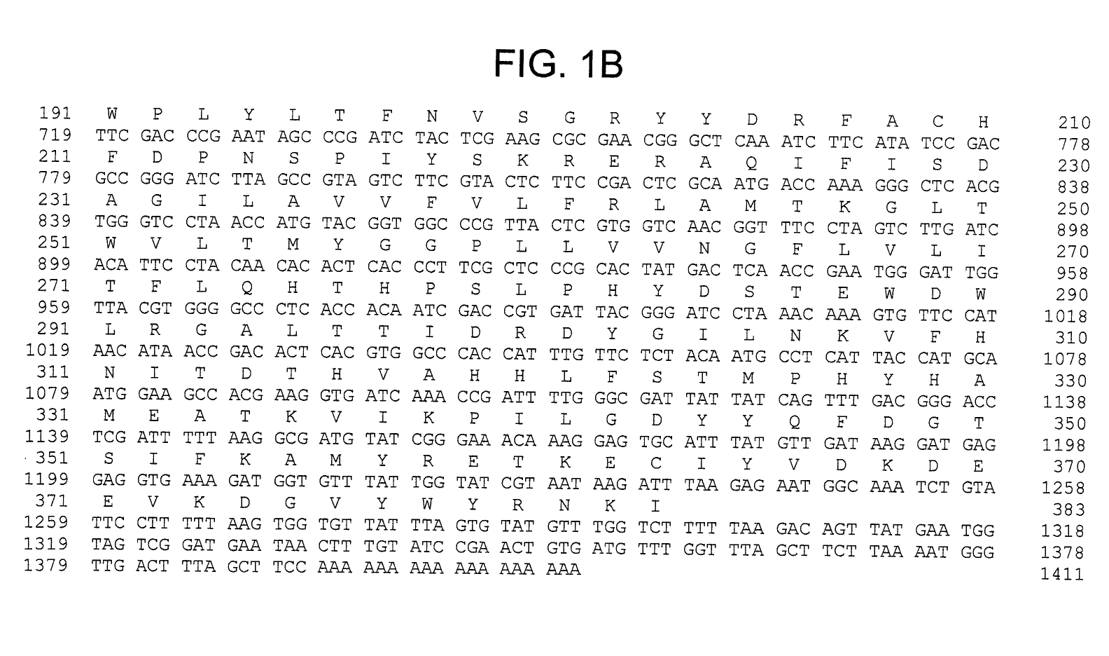

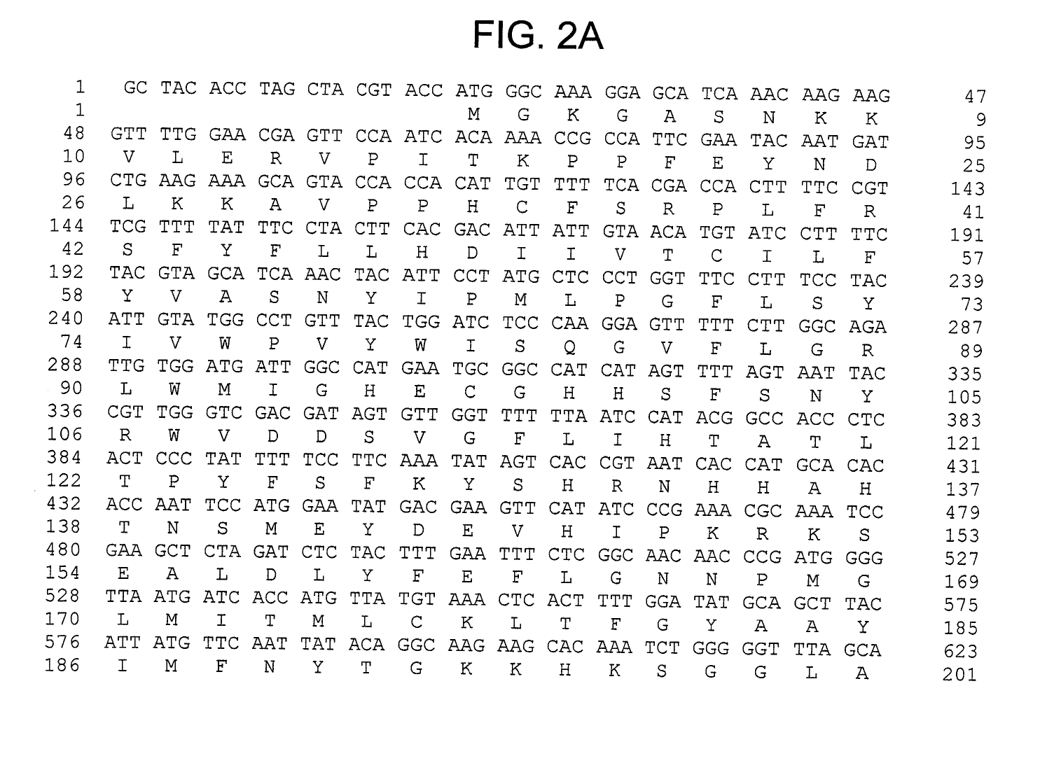

To isolate full-length cDNA clones, the two types of Fad2-like inserts were used as probes to screen a cDNA library from developing seeds, which resulted in identification of several cDNA clones in each group. Sequencing identified two unique full-length of cDNAs, CoFad2 and CoFac2. CoFad2 is 1411 bp and codes for 383 amino acids with molecular weight of 44 kDa (FIG. 1 and SEQ ID Nos: 3 and 4). CoFac2 is 1301 bp in length and codes for 374 amino acids with molecular weight of 43.6 kDa (FIG. 2 and SEQ ID Nos: 1 and 2). Sequence comparison revealed 46% amino acid identity between the two deduced proteins. The identity occurs all along the polypeptides with the highest among three conservative histidine-rich areas (FIG. 3).

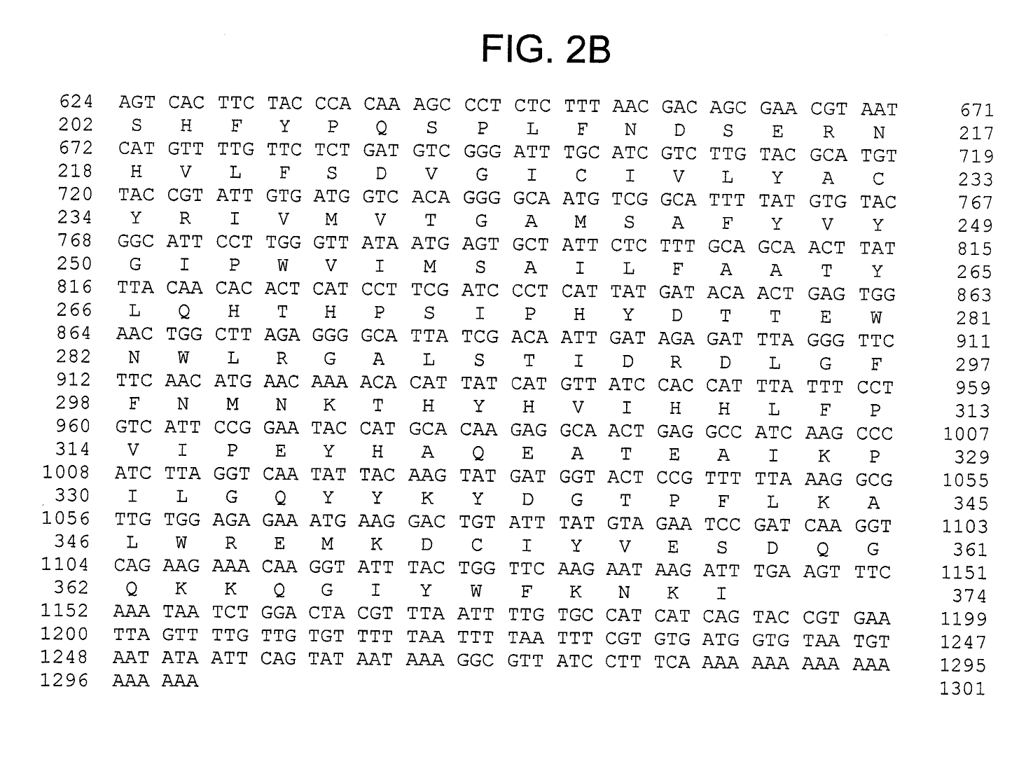

Sequence comparisons indicate that CoFad2 (SEQ ID Nos: 3 and 4) shares 73-89% amino acid identity with the Δ12 desaturases from various plants. CoFac2 (SEQ ID Nos: 1 and 2) shares approximately equal sequence identity (50%) to both FAD2 desaturases and related enzymes, including FAD2 from Calendula (SEQ ID NOs:3 and 4), Brassica juncea and borage, the Δ12 acetylenase of Crepis alpina, the bifunctional enzyme (oleate 12-hydroxylase: 12-desaturase) of Lesquerella fendleri, the 12,13-epoxygenase of Crepis palaestina, as well as various other genes reported to encode fatty acid conjugases from Calendula officinalis, Impatiens balsamina and Momordica charantia. Cluster analysis indicated that CoFac2 distinguished itself from homologous enzymes and formed a separate group by itself, although it is relatively closer to Crepis palaestina epoxgenase rather than the two congjugases. CoFad2 is more closely related to Lesquerella fendleri bifunctional enzyme and Ricinus communis hydroxylase (FIG. 4).

These results suggest that CoFad2 may function as a extraplastidial Δ12 fatty acid desaturase and CoFac2 may function as a fatty acid modifier likely to be involved in calendic acid biosynthesis.

### Example 3

### Characterization of CoFac2 and CoFad2

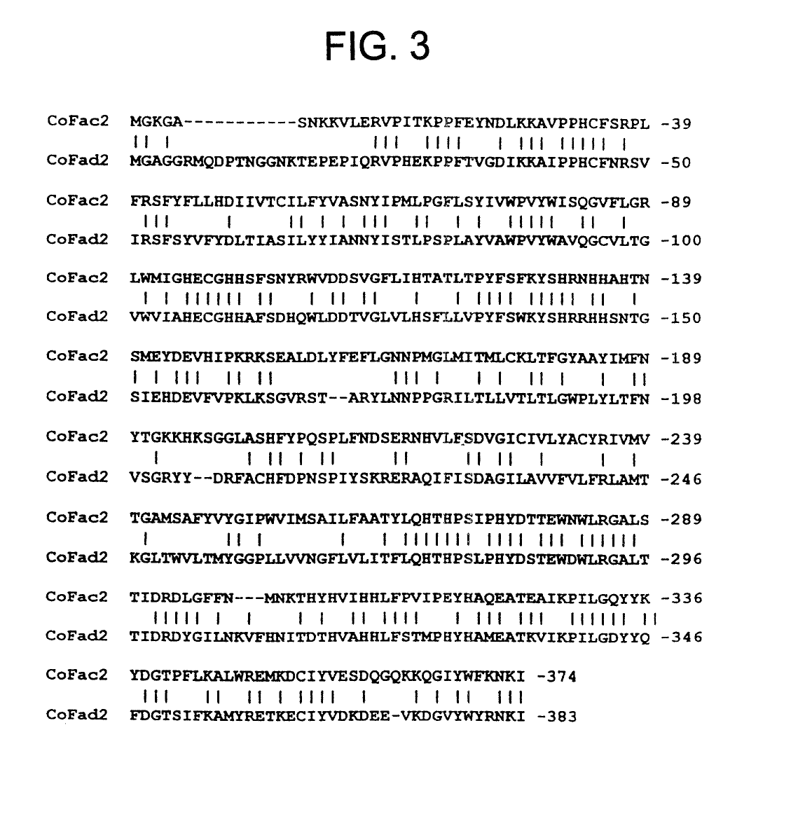

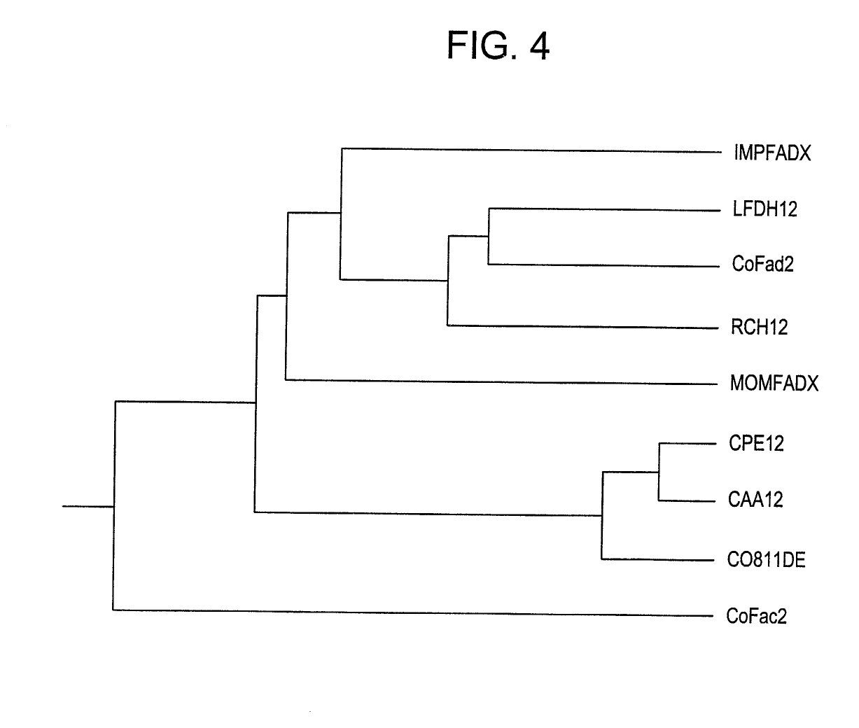

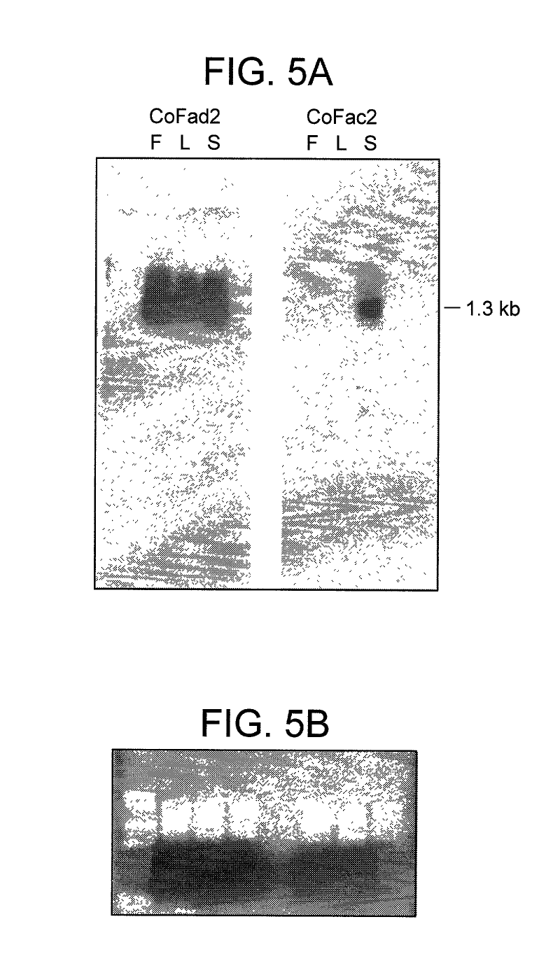

Northern blot analysis indicated that the CoFac2 was exclusively expressed in the developing seeds of C. officinalis (FIG. 5). It was not expressed in vegetative tissues such as leaves, and reproductive tissues such as flower buds. In contrast, CoFad2 was expressed in all tissues tested such as leaves, flower buds and developing seeds, but preferentially in flower buds and developing seeds. Expression patterns of the two genes were consistent with the pattern of calendic acid accumulation, which occurs only in seeds. As set forth in Example 1, above, in C. officinalis calendic acid accumulated only in seeds, whereas linoleic acid, the product of the Δ12 desaturase (CoFAD2) was present in all three tissues examined although the flower buds and developing seeds contain a higher amount linoleic acid (see Table 1).

### EXAMPLE 4

### Expression of CoFac2 and CoFad2 in Saccharomyces cerevisiae

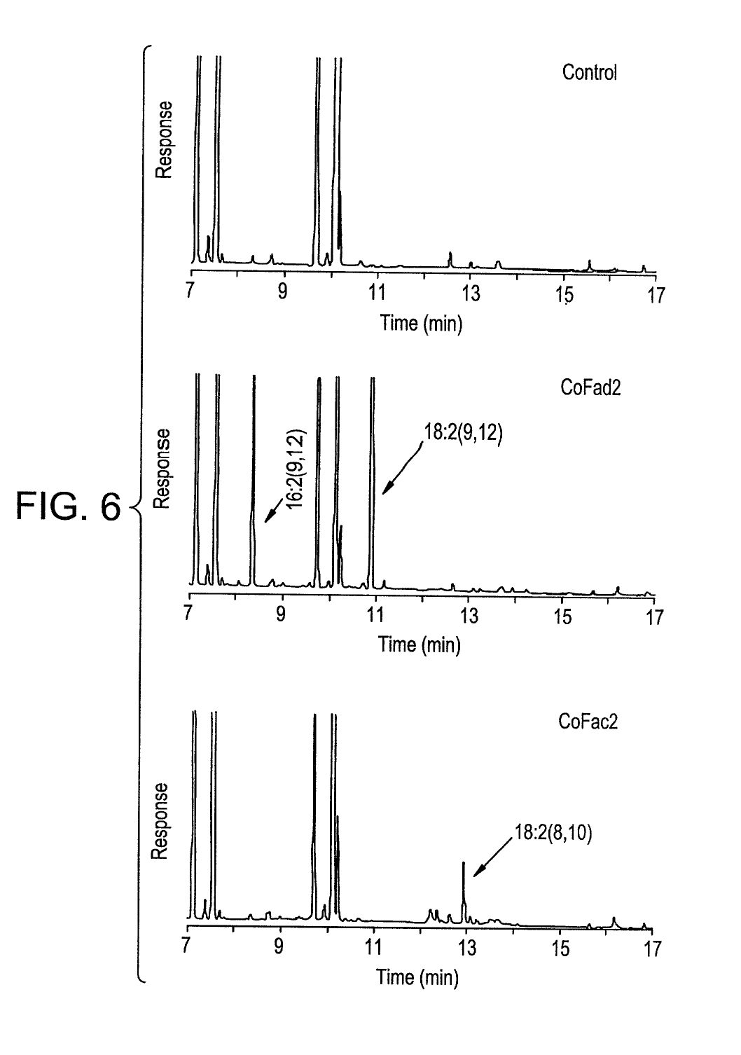

To confirm the function of the two genes, both full-length cDNAs were expressed in yeast strain Invsc2 under the control of the inducible promoter. FIG. 6 shows that without supplementation of any exogenous substrates, both clones could convert endogenous fatty acids into respective products. As compared to the control that (containing cloning vector only), yeast cells containing CoFad2 cDNAs were observed to have two extra peaks in the chromatogram of fatty acid methyl esters, which had retention times identical to the 16:2 (6, 9) and 18:2 (6, 9) standards, respectively, indicating that CoFad2 is an authentic Δ12 desaturase which was able to introduce a double bond at 12 position of palmitoleic (16: 1, 9) and oleic (18:1, 9) acids. As compared to the control, yeast transformant containing CoFac2 cDNA was found to have one major extra peak with retention time 13.48 min in the chromatogram.

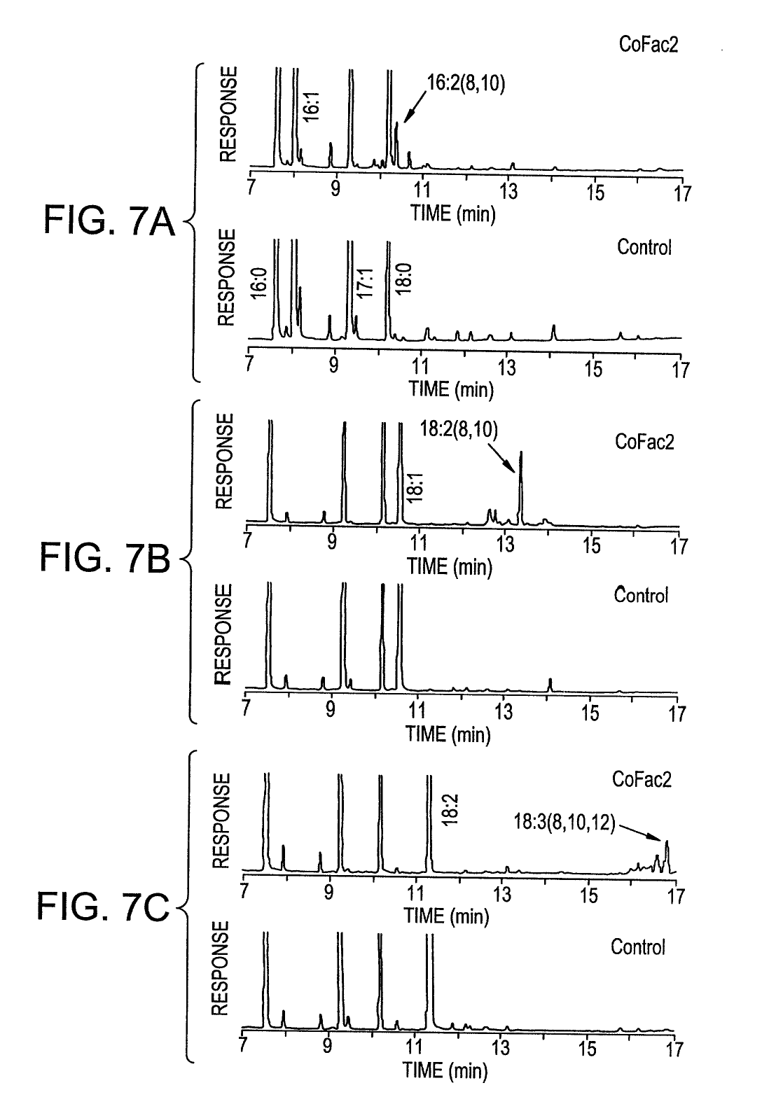

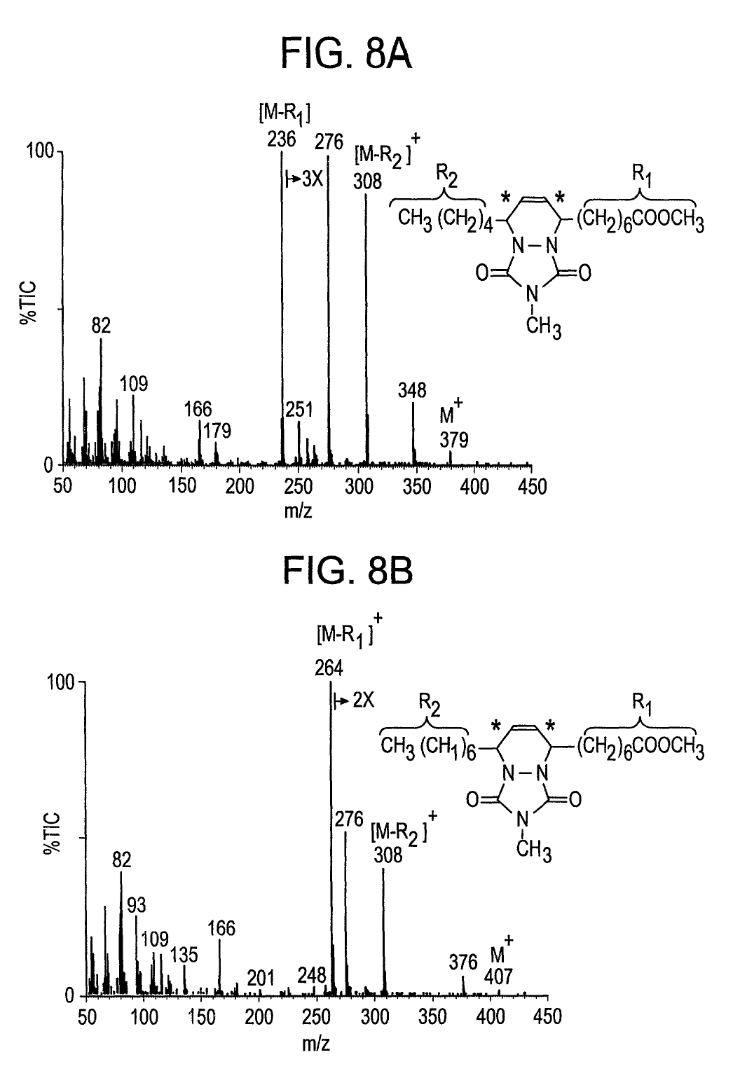

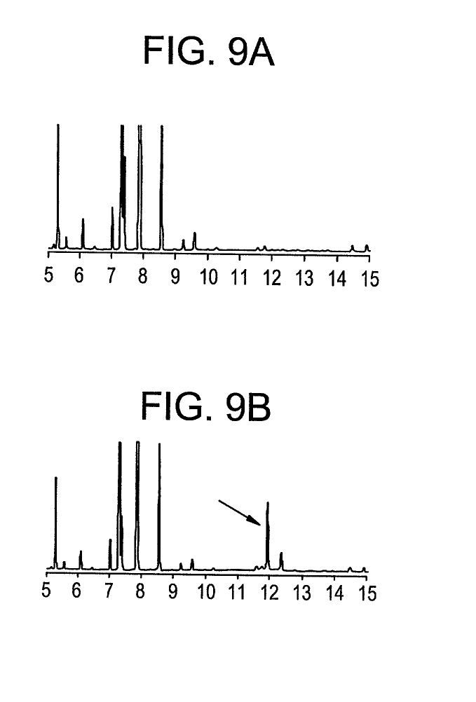

To investigate substrate specificity of CoFac2, the full-length cDNA was expressed in the yeast strain AMY-2α in which the stearoyl-CoA desaturase gene, ole1, is disrupted. The strain is unable to grow in minimal media without supplementation of mono-unsaturated fatty acids and allows for experimental control of the fatty composition of the yeast. In our experiments, the strain was grown in minimal medium supplemented with 17:1 (10Z), a non-substrate of CoFAC2, which enabled us to study the substrate specificity of the enzyme towards various substrates, especially monounsaturates. A number of possible substrates including 16:0, 16:1(9Z), 17:1(10Z), 18:0, 18:1(9Z), 18:1(9E), 18:1(11Z), 18:1(11E), 18:1(12Z), 18:1(15Z), 18:2(9Z,12Z), 18:3(9Z,12Z,15Z), 20:0, 20:2(l1 Z,14Z) and 22:1(13Z) were tested. As indicated in FIG. 7, only 18:2(9Z,12Z), 16:1(9Z) and 18:1(9Z) were converted to conjugated fatty acids by the enzyme. For cultures supplemented separately with the three substrates, when gas chromatograms of FAME derived from strains expressing CoFac2 were compared with those for vector controls, extra peaks were detected as shown in FIG. 7. These peaks were selectively ablated when a Diels-Alder reaction with MTAD was performed prior to GC analysis. The sets of m/z peaks indicated in FIG. 8 are highly diagnostic for the original double bond positions of the conjugated fatty acid analyte. Mass spectral analysis of the MTAD derivatives indicates that the products of 16:1, 18:1 and 18:2 conversion are 16:2(8,10), and 18:2(8,10) (FIG. 9) and 18:3(8,10,12). Assignment of the product of 18:1(9) conversion is also supported by the agreement of its GC peak retention time with one of a mixture of standard CLA isomers (data not shown). The mass spectrum for the analyte identified as 18:3(8,10,12) is consistent with two compounds for which the Diels-Alder reaction has occurred either at the 8 and 10 positions, or the 10 and 12 positions of an 18:3 isomer. This compound has the same GC retention time as the major FAME derived from Calendula seeds and is in all likelihood 18:3(8E,10E,12Z). In control experiments with the AMY2α/pYES2 strain, no peaks corresponding to conjugated fatty acids were detected.

### Example 5

### Expression of CoFac2 in Brassica juncea

To determine whether CoFac2 is functional in oilseed crops, we transformed B. juncea with the construct containing CoFac2 under the control of the napin promoter. Four independent transgenic plants were obtained. In the gas chromatogram of transgenic seeds, a new fatty acid appeared which has retention time identical to calendic acid (FIG. 10) indicating that CoFac2 is functional in oilseed crops.

### Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule which encodes a polypeptide having an activity of catalyzing the formation of two conjugated double bonds, wherein said nucleic acid molecule comprises:
a. a nucleotide sequence of CoFac2 from the genus Calendula;
b. a nucleotide sequence which is at least about 60% identical to the nucleotide sequence of SEQ ID NO:1, or a complement thereof;
c. a nucleotide sequence comprising a fragment of the nucleotide sequence of SEQ ID NO:1;
d. a nucleotide sequence which encodes a polypeptide comprising an amino acid sequence that is at least about 60% homologous to the amino acid sequence of SEQ ID NO:2;
e. a nucleotide sequence which encodes a fragment of a polypeptide comprising the amino acid sequence of SEQ ID NO:2, wherein the fragment comprises at least 15 contiguous amino acids of SEQ ID NO:2; or
f. a nucleotide sequence which encodes a naturally occurring allelic variant of a polypeptide comprising the amino acid sequence of SEQ ID NO:2, wherein the nucleic acid molecule hybridizes to a nucleic acid molecule comprising SEQ ID NO:1, or a complement thereof under stringent conditions.

2. The isolated nucleic acid molecule of claim 1, wherein the nucleic acid molecule encodes a polypeptide having an activity of catalyzing the formation of two conjugated double bonds at carbon positions 8 and 10, numbered from the carboxyl end of a fatty acyl chain.

3. An isolated nucleic acid molecule which encodes a protein having an activity of catalyzing the formation of a double bond, wherein said nucleic acid molecule comprises:
a. a nucleotide sequence of CoFad2 from the genus Calendula;
b. a nucleotide sequence which is at least about 60% identical to the nucleotide sequence of SEQ ID NO:3, or a complement thereof;
c. a nucleotide sequence comprising a fragment of the nucleotide sequence of SEQ ID NO:3;
d. a nucleotide sequence which encodes a polypeptide comprising an amino acid sequence that is at least about 60% homologous to the amino acid sequence of SEQ ID NO:4;
e. a nucleotide sequence which encodes a fragment of a polypeptide comprising the amino acid sequence of SEQ ID NO:4, wherein the fragment comprises at least 15 contiguous amino acids of SEQ ID NO:4; or
f. a nucleotide sequence which encodes a naturally occurring allelic variant of a polypeptide comprising the amino acid sequence of SEQ ID NO:4, wherein the nucleic acid molecule hybridizes to a nucleic acid molecule comprising SEQ ID NO:3, or a complement thereof under stringent conditions.

4. An isolated nucleic acid molecule of claim 2, wherein the nucleic acid molecule encodes a protein having an activity of catalyzing the formation of a double bond at position 12 numbered from the carboxyl end of a fatty acyl chain.

5. A vector comprising the nucleic acid molecule of any one of claims 1 and 3.

6. A cell transformed with the nucleic acid molecule of any one of claims 1 and 3.

7. The cell of claim 6, wherein said cell is a plant cell.

8. A plant transformed with the nucleic acid molecule of any one of claim 1 or 3.

9. A method of producing a cell capable of generating conjugated linoleic acid or linolenic acid, said method comprising introducing into said cell the nucleic acid molecule of claim 1, wherein the nucleic acid molecule encodes a conjugase having an activity of catalyzing the formation of two conjugated double bonds at position 8 and 10 numbered from the carboxyl end of a fatty acyl chain.

10. The method of claim 9, wherein said cell is a plant cell.

11. A method of producing a cell capable of producing linoleic acids, said method comprising introducing into said cell the nucleic acid molecule of claim 3, wherein the nucleic acid molecule encodes a protein having an activity of catalyzing the formation of a double bond at position 12 numbered from the carboxyl end of a fatty acyl chain

12. The method of claim 11, wherein said cell is a plant cell.

13. A method for modulating the production of fatty acids comprising culturing a cell as claimed in claim 6, such that modulation of fatty acid production occurs.

14. The method of claim 13, wherein the production of fatty acids is enhanced.

15. The method of claim 13, wherein the fatty acid produced is conjugated linoleic acid.

16. The method of claim 13, wherein the fatty acid produced is linolenic acid.

17. The method of claim 13, wherein the fatty acid produced is calendic acid.

18. The method of claim 13, further comprising recovering the fatty acid produced.

19. A method for large scale production of CFAs, comprising culturing a cell as claimed in claim 6, such that a conjugated fatty acid is produced.

20. The method of claim 19, wherein the production of conjugated fatty acids is enhanced.

21. The method of claim 19, wherein the conjugated fatty acid produced is conjugated linoleic acid.

22. The method of claim 19, wherein the conjugated fatty acid produced is linolenic acid.

23. The method of claim 19, wherein the conjugated fatty acid produced is calendic acid.

24. The method of claim 19, further comprising recovering the conjugated fatty acid produced.

25. A linoleic acid composition containing an effective amount of linoleic acids having two conjugated double bonds at position 8 and 10 numbered from the carboxyl end of a fatty acyl chain.

26. The composition of claim 25, wherein said composition is administrated to humans or animals.

27. The composition of claim 25, wherein said composition is used for cosmetic or pharmaceutical purposes.

28. A method of producing 8 carbon and 10 carbon alkanes, comprising breaking the double bonds of conjugated linoleic acids as defined in claim 25.

29. A method of using conjugated linoleic acids as defined in claim 25 for coating, painting or cold weather ester-type lubricants purposes.

30. A linolenic acid composition containing an effective amount of linolenic acids having three conjugated double bonds at position 8, 10 and 12 numbered from the carboxyl end of a fatty acyl chain.

31. The composition of claim 30 wherein said composition is used for cosmetic or pharmaceutical purposes.

32. A method of producing 4, 8 carbon and 10 carbon alkanes, which comprises breaking the double bonds of conjugated linolenic acids defined in claim 30.

33. A method of using conjugated linolenic acids as defined in claim 30 for coating, painting or cold weather ester-type lubricants purposes.
